(19) United States
(12) Patent Application Publication
(10) Pub. No.: US 2025/0000466 A1
(43) Pub. Date: Jan. 2, 2025

(54) METHOD FOR LOCALIZING A TOOL IN AN X-RAY IMAGE, DATA PROCESSING APPARATUS, X-RAY IMAGING SYSTEM, AND COMPUTER PROGRAM PRODUCT

(71) Applicant: Siemens Healthineers AG, Forchheim (DE)

(72) Inventors: Alois Regensburger, Poxdorf (DE); Christian Kaethner, Freiburg (DE)

(21) Appl. No.: 18/753,115

(22) Filed: Jun. 25, 2024

(30) Foreign Application Priority Data
Jun. 29, 2023 (DE) 10 2023 206 148.1

(51) Int. Cl. A61B 6/12; A61B 6/00; A61B 6/46; A61B 6/58; A61B 34/20; A61M 25/01

(52) U.S. Cl. CPC A61B 6/12; A61B 6/463; A61B 6/5217; A61B 6/582; A61B 34/20; A61B 2034/2065; A61M 2025/0166

(57) ABSTRACT

For localizing a tool in an X-ray image, wherein the tool has an X-ray absorption strength spatially changeable along a specified longitudinal direction of the tool according to a specified absorption characteristic, a spatial frequency spectrum of a portion of the X-ray image is determined and it is checked whether the spatial frequency spectrum corresponds to the absorption characteristic of the tool. If it is established that the spatial frequency spectrum corresponds to the absorption characteristic of the tool, the portion of the X-ray image is determined as the position of the tool.

The present patent document claims the benefit of German Patent Application No. 10 2023 206 148.1, filed Jun. 29, 2023, which is hereby incorporated by reference in its entirety.

### TECHNICAL FIELD

The present disclosure relates to a method for localizing a tool in an X-ray image, to an associated data processing apparatus for carrying out such a method, to an X-ray imaging system with such a data processing apparatus, and to a corresponding computer program product.

### BACKGROUND

In medical interventions, in particular minimally invasive interventions, fluoroscopy techniques may be used in which the intervention may be visually represented and followed using X-ray-based imaging. During such interventions, tools are introduced into the body of the patient, which are likewise visible on the fluoroscopy X-ray images and may be tracked accordingly by the person carrying out the treatment. In the field of angiography, such tools may include catheters that are introduced into hollow organs, in particular blood vessels.

The situation may occur with very small catheters or catheters with very small diameters, (e.g., what are known as microcatheters with diameters at the tip of less than 2 mm or less than 1 mm), wherein the catheter is difficult to see or may barely be seen in the X-ray images by an observer.

In order to improve the visibility of such tools in X-ray images, the X-ray dose used may be increased and/or the image rate when acquiring the X-ray images may be increased. However, this would be accompanied by increased exposure to radiation for the patient and for other individuals in their environment.

It would also be possible to carry out digital subtraction angiography in order to suppress potentially irrelevant backgrounds in the X-ray images and to highlight, inter alia, the tool more accordingly. However, this results in considerably more effort during image processing.

The use of dual energy X-ray acquisition methods would also be conceivable but would likewise be connected with increased effort during image processing and, sometimes, increased exposure to radiation.

### SUMMARY AND DESCRIPTION

It is an object of the present disclosure to simplify the localization of a tool in an X-ray image, in particular without increasing the exposure to radiation. The scope of the present disclosure is defined solely by the appended claims and is not affected to any degree by the statements within this summary. The present embodiments may obviate one or more of the drawbacks or limitations in the related art.

The disclosure is based on the idea of providing a tool, in particular a catheter, which has a spatially changeable X-ray absorption strength along a specified longitudinal direction of the tool according to a specified absorption characteristic. By analyzing the corresponding spatial frequency spectrum in the resulting X-ray image, it is possible to establish where the tool is located in the X-ray image, or whether the tool is located in a particular portion of the X-ray image.

According to one aspect, a method, (e.g., a computer-implemented method), for localizing a tool in an X-ray image is disclosed. The tool has a spatially changeable X-ray absorption strength along a specified longitudinal direction of the tool according to a specified absorption characteristic. In particular, the tool is specified or provided such that it has the spatially changeable X-ray absorption strength according to the specified absorption characteristic.

A spatial frequency spectrum of a portion of the X-ray image is determined, in particular by at least one computing unit, for example, automatically. It is checked, in particular by the at least one computing unit, (e.g., automatically), whether the spatial frequency spectrum corresponds to the absorption characteristic of the tool. If it is established when checking that the spatial frequency spectrum corresponds to the absorption characteristic of the tool, the portion of the X-ray image is determined as the position of the tool, in particular by the at least one computing unit, for example, automatically.

Unless stated otherwise, all acts of the computer-implemented method may be carried out by a data processing device that has the at least one computing unit. In particular, the at least one computing unit is configured or adapted for carrying out the acts of the computer-implemented method. For this, the at least one computing unit may store, for example, a computer program that includes commands, which, on execution by the at least one computing unit, prompt the at least one computing unit to execute the computer-implemented method.

For each embodiment of the computer-implemented method, a corresponding embodiment of a method for localizing a tool, which is not purely computer-implemented, results directly in that a method act is encompassed according to which the X-ray image is generated.

The at least one computing unit may be part of an X-ray imaging system, in particular an X-ray angiography system.

For example, the X-ray image may represent the tool as well as an organ, (e.g., a hollow organ such as a blood vessel or a vascular tree), of the patient. The tool may be a catheter, in particular a microcatheter. The catheter may then be introduced on the X-ray image, in particular into the hollow organ.

Reference may be made to the fact that interventional acts, (e.g., the introduction of the tool into the body of the patient or guiding of the tool inside the body of the patient), are potentially not part of the method.

The X-ray image may be a two-dimensional X-ray image or an X-ray projection image. Such X-ray images have the advantage, in contrast to three-dimensional reconstructions, that they may be generated quickly during the intervention and with low radiation doses. The visibility of the tool in the X-ray image naturally suffers when using lower radiation doses, however, so the disclosure has a particularly advantageous impact here.

The X-ray image corresponds to a spatial, in particular two-dimensional, distribution of an image intensity or image brightness, which are given by the corresponding pixel values of the X-ray image. In other words, the X-ray image therefore reproduces an intensity in the spatial domain. An analog representation, although less intuitive for human observers, results in what is known as the frequency domain or spatial frequency domain, which may be regarded as the reciprocal domain of the spatial domain. The spatial domain and the frequency domain are linked together, in particular, by a Fourier transform.

In other words, variations in brightness in the X-ray image may also be represented by corresponding frequency components of a spectrum in the frequency domain. The disclosure accordingly uses the spatially changeable X-ray absorption strength of the tool along the longitudinal direction in order to see it in the frequency domain of the representation. Because the spatial frequency spectrum is determined for a given portion of the X-ray image in the present case, by identifying the tool using its absorption characteristic in the frequency domain, it is also possible to localize its position in the spatial domain, namely on the examined or analyzed portion of the X-ray image. Localizing the tool may therefore include ascertaining a particular portion of the X-ray image, within which the spatial frequency spectrum corresponds to the absorption characteristic of the tool.

There are various possibilities for determining the spatial frequency spectrum not for the entire X-ray image, but only for the portion that is, in particular, smaller than the entire X-ray image. For this, the X-ray image may be divided, for example, into predefined cells or regions of interest, with each region of interest corresponding to a corresponding portion of the X-ray image and the spatial frequency spectrum is ascertained accordingly for the individual regions of interest and is compared with the absorption characteristic of the tool.

It is also possible in the case of the Fourier transform from the spatial domain into the frequency domain to convolute the corresponding Fourier operator with a filter operator, in particular a local filter operator. The filter operator then ensures that with a given position of the filter operator in the X-ray image, only items of image information from a predefined region contribute, or significantly contribute, to the Fourier transform or, in other words, items of image information outside of the corresponding region are strongly suppressed. It may be a Gaussian filter or the like.

In particular, the tool, (e.g., the catheter), has a rigid region that extends along the longitudinal direction. The spatially changeable X-ray absorption strength is accordingly present in the rigid part. That the rigid part extends along the longitudinal direction may be taken to mean, in particular, that a spatial extent of the rigid part is a lot greater along the longitudinal direction than a spatial extent perpendicular to the longitudinal direction, with it being possible for “a lot greater” to correspond, for example, to a factor of at least 10, at least 50, at least 100, in a range of 10 to 200, or the like. In other words, a spatial extent of the tool in the region of the rigid part along an arbitrary direction perpendicular to the longitudinal direction is smaller by at least the factor than a length, e.g., a spatial extent of the rigid part along the longitudinal direction.

The spatially changeable X-ray absorption strength may be achieved, for example, by using different materials for the tool or attaching different materials to a surface of the tool. For example, a material composition of the tool may vary along the longitudinal direction accordingly. A tool, such as a catheter, may include various metals, metal weaves, composite materials, composite weaves, and/or composite fiber materials, etc. The composite materials, composite weaves, and composite fiber materials may include metals, plastics materials, and/or carbon fibers, etc. An appropriate absorption characteristic of the tool may be specified by purposeful modification or modulation of the material composition along the longitudinal direction.

The absorption characteristic of the tool therefore corresponds, for example, to a reference spatial frequency spectrum of the spatial change in the X-ray absorption strength along the longitudinal direction. In order to check whether the spatial frequency spectrum of the portion corresponds to the absorption characteristic of the tool, the absorption characteristic, (e.g., the reference spatial frequency spectrum), may be compared with the spatial frequency spectrum of the portion.

In particular, certain frequencies in the reference spatial frequency spectrum may be purposefully highlighted in that the X-ray absorption strength of the tool along the longitudinal direction is periodically or quasi-periodically specified for two or more period durations. In a non-limiting illustrative example, the absorption characteristic of the tool may correspond to a periodic change with a single defined frequency, for example, a sinusoidal characteristic curve or a characteristic curve of a rectangular curve, etc. In this case, it would be possible to see a pronounced peak, also referred to as a peak value, in the reference spatial frequency spectrum in the case of the corresponding fundamental frequency and potentially, for example, in the case of a periodic rectangular signal, at corresponding higher harmonic frequencies.

If the tool is in the examined portion, then corresponding peaks will also result in the spatial frequency spectrum of the portion, so the comparison may be positive in this case, whereas a corresponding match may not be found in other portions. Reference may be made in this connection to the fact that the absolute position of the peak in the spatial frequency spectrum of the portion, even if the tool is located in the portion, does not necessarily exactly match the corresponding peak in the reference spatial frequency spectrum.

This may be the case, in particular, if the longitudinal direction does not run parallel to the X-ray projection plane of the X-ray image but encloses therewith an angle different from zero. Such angle variations may result in shifts in the frequency spectrum. For example, the orientation of the tool in the three-dimensional space may potentially be estimated or at least localized, however, by further items of information, so a corresponding comparison is still possible. This may take place, for example, on the basis of pre-operative three-dimensional CT reconstructions or the like or on the basis of anatomical boundary conditions that result in it only being possible for the tool to be located in particular orientations or, in any event, approximately in such orientations.

The X-ray projection plane is a plane that runs parallel to a detector plane of an X-ray detector with which the X-ray image was generated and is perpendicular to the central X-ray projection beam. Consequently, only the orientation of the X-ray projection plane is clearly defined, and not its location, although the location may be arbitrarily selected because it is only a matter of the orientation of the X-ray projection plane.

Further, it is possible, when using cone beam geometries, for the peaks to shift by the spacing of the tool from the X-ray source.

Furthermore, appropriate selection of the frequency ranges, in which peaks are found in the absorption characteristic, may still make clear identification possible if, for example, no other peaks are to be expected in the corresponding frequency range. Furthermore, the absorption characteristic may also be more complex and define, for example, a sequence of two or more peaks in the reference spatial frequency spectrum. If, accordingly, there is likewise a sequence of a corresponding number of two or more peaks in the spatial frequency spectrum of the portion, this may serve localization of the tool even if the exact positions of the peaks and potentially their spacings differ due to different orientations of the tool in respect of the X-ray projection plane.

It is also possible to estimate the angle in that two or more X-ray images with different X-ray projection directions are analyzed and to determine differences in the peak positions or the like in different X-ray images. The angle may then be estimated on the basis of this. Optionally, the angle may also be corrected or the location of the X-ray imaging system, for instance of an X-ray source and an X-ray detector of the X-ray imaging system, may be purposefully adjusted in order to reduce the angle.

The portion is, in particular, a two-dimensional portion of the X-ray image. The spatial frequency spectrum may also be a two-dimensional spatial frequency spectrum accordingly. The corresponding absorption characteristic of the tool may then be compared with the two-dimensional spatial frequency spectrum. In particular, it is not necessary to know in advance for this how the longitudinal direction or its projection is oriented inside the X-ray projection plane. It is also possible, however, for the spatial frequency spectrum to be calculated as a one-dimensional spatial frequency spectrum and to be analyzed for various directions accordingly.

The disclosure utilizes the specific spatial variability of the X-ray absorption strength of the tool along the longitudinal direction in order to localize it in the X-ray image. Consequently, the measures mentioned in the introduction for improving the visibility may be omitted, and this overcomes the drawbacks. It is also possible, however, to combine the method with one or more of the measure(s) mentioned in the introduction in order to guarantee even better visibility. The drawbacks mentioned in the introduction may then potentially be at least partially overcome or the visibility may be improved further compared to known approaches.

According to at least one embodiment, according to the absorption characteristic, the spatial change in the X-ray absorption strength recurs along the longitudinal direction or up to a differing amplitude.

That the spatial change recurs may be taken to mean, in particular, that the X-ray absorption strength is in each case identical in at least two consecutive lengths along the longitudinal direction. That the spatial change in the X-ray absorption strength recurs up to a differing amplitude, may be taken to mean that the X-ray absorption strengths during two consecutive lengths are identical up to a constant factor in the amplitude or a linear position-dependent factor or a factor modulated in some other way, a factor modulated in a defined manner.

In particular, the tool is accordingly provided such that the spatial change in the X-ray absorption strength along the longitudinal direction according to the absorption characteristic recurs or recurs up to the differing amplitude.

The recurrence, in particular at least up to the differing amplitude, means that peaks pronounced in the reference spatial frequency spectrum are present at corresponding points. This facilitates a comparison with the spatial frequency spectrum of the portion.

According to at least one embodiment, the spatial change in the X-ray absorption strength along the longitudinal direction according to the absorption characteristic has a characteristic frequency at which the reference spatial frequency spectrum of the spatial change in the X-ray absorption strength along the longitudinal direction has a peak value. In order to check whether the spatial frequency spectrum corresponds to the absorption characteristic of the tool, it is checked whether the spatial frequency spectrum has a peak value at a point that corresponds within a specified tolerance range to the characteristic frequency.

In particular, the tool is accordingly provided such that the spatial change in the X-ray absorption strength along the longitudinal direction according to the absorption characteristic has the characteristic frequency.

A peak value may, as already mentioned above, also be referred to as a peak. Known algorithms for peak finding may be used to check the presence of corresponding peak values in the spatial frequency spectrum of the portion. Suitable parameters to be varied, (e.g., a peak form, peak height, peak width, etc.), may be specified for this.

As also already mentioned above, the characteristic frequency is not necessarily identical to the point in the spatial frequency spectrum at which the peak value is to be expected if the tool is located in the portion. This may be attributed in part to the orientation of the longitudinal direction in respect of the X-ray projection plane. The tolerance range may take such deviations into account in that it is selected, in particular, using empirical values or other boundary conditions. If the orientation of the longitudinal direction may be localized, for example, to a particular angle range, for example, on the basis of pre-operative imaging or other boundary conditions, then the tolerance range may be restricted accordingly.

According to at least one embodiment, a similarity measure is calculated between the spatial frequency spectrum and the reference spatial frequency spectrum in order to check whether the spatial frequency spectrum corresponds to the absorption characteristic of the tool.

The calculated value of the similarity measure is compared, for example, with a specified threshold value. If the calculated value of the similarity measure is greater than the threshold value, then it may be established, for example, that the spatial frequency spectrum corresponds to the absorption characteristic of the tool.

The similarity measure may be calculated, for example, as a cross-correlation or other overlap integral.

Further embodiments of the method may also be found from the embodiment for further characteristic frequencies that may be dealt with analogously.

The tolerance range may correspond, for example, to a range [f0/(1+cos(α)), f0/(1−cos(α′))], wherein f0 corresponds to the characteristic frequency according to the absorption characteristic, and the angles α and α′ correspond to the maximum expected deviations of the longitudinal direction from the X-ray projection plane.

An automated simple comparison of the spatial frequency spectrum with the absorption characteristic may be achieved in this way.

According to at least one embodiment, the spatial change in the X-ray absorption strength along the longitudinal direction according to the absorption characteristic has two characteristic frequencies at which the reference spatial frequency spectrum of the spatial change in the X-ray absorption strength along the longitudinal direction has one peak value respectively. In order to check whether the spatial frequency spectrum corresponds to the absorption characteristic of the tool, it is checked whether, within a specified tolerance range, the spatial frequency spectrum, at two points, whose spacing from one another corresponds to a spacing of the two characteristic frequencies from one another, has one peak value respectively.

Such embodiments may potentially be expanded by further peak values and corresponding spacings therebetween. It is also possible to combine such embodiments, in which the spacing of the characteristic frequencies is analyzed, with the embodiments mentioned above in which the point of the characteristic frequency is analyzed.

Analogously to as described above, the tolerance range for the spacing df also results, for example, from the angles α and α′ that are the maximum ones to be expected between the longitudinal direction and the X-ray projection direction, so [df/(1+cos(α)), df/(1−cos(α′))].

In particular, the tool is accordingly provided such that the spatial change in the X-ray absorption strength along the longitudinal direction according to the absorption characteristic has the two characteristic frequencies.

An automated simple comparison of the spatial frequency spectrum with the absorption characteristic may be achieved in this way.

According to at least one embodiment, in order to check whether the spatial frequency spectrum corresponds to the absorption characteristic of the tool, it is checked whether a relationship of the peak values at the two points of the spatial frequency spectrum corresponds to a relationship of the peak values in the case of the two characteristic frequencies of the reference spatial frequency spectrum.

It is therefore not just the relative position of the two characteristic frequencies that is checked but also their relative amplitude in respect of one another. It is also possible that, in other embodiments, only the relationship of the peak values is checked and not the spacing between the characteristic frequencies.

When checking whether the relationship of the peak values of the spatial frequency spectrum corresponds to the relationship of the peak values of the characteristic frequencies of the reference spatial frequency spectrum, for example, a specified or predetermined modulation transfer function (MTF) may be taken into account. The modulation transfer function describes, in particular for a given X-ray imaging system or a complete image chain of the X-ray source used, or the spectrum of the emitted X-ray radiation, via the object, the X-ray detector, the signal processing and the image display, how a given frequency in the object domain is transferred or mapped to a frequency in the image domain in the X-ray image. The MTF is a characteristic of the X-ray imaging system used or the image chain.

Along the image chain, higher frequencies may be attenuated more or suppressed more than lower frequencies. This may be expressed by a corresponding MTF, which has a lower value in the case of higher frequencies than in the case of lower frequencies. The MTF may be determined by calibration measurements or the like. The reference spatial frequency spectrum of the spatial change in the X-ray absorption strength may then be given, for example, as a convolution of the modulation transfer frequency with an original reference spectrum, with the original reference spectrum resulting from the Fourier transform of the spatial change in the X-ray absorption strength without taking into account the MTF.

The MTF may also be indirectly taken into account, however, in that, for example, reference mappings of the tool are created against a neutral background and the reference spatial frequency spectrum is determined by corresponding Fourier transform.

According to at least one embodiment, a further X-ray image is generated that represents the tool in a calibration environment, e.g., against a neutral background. The absorption characteristic, in particular the reference spatial frequency spectrum, is determined on the basis of the further X-ray image.

The corresponding effects of the image chain or the MTF may be indirectly easily taken into account in this way, and this results in increased reliability of the method.

According to at least one embodiment, an angle β, which the longitudinal direction of the tool encloses with a specified X-ray projection plane when generating the X-ray image, in particular an X-ray projection plane corresponding to the X-ray image on the generation thereof, is obtained or determined, in particular by the at least one computing unit. The check as to whether the spatial frequency spectrum of the portion corresponds to the absorption characteristic of the tool is carried out as a function of the angle β.

If, for example, for the case where the longitudinal direction is parallel to the X-ray projection plane, a peak or a spacing of two peaks from one another according to the absorption characteristic is expected, then a shift of the peak(s) f→cos(β)*f or a compression of the spectrum A(f), so A(f)→A(cos(β)*f) results.

According to at least one embodiment, the X-ray image maps a hollow organ, wherein the tool is arranged in the hollow organ.

The hollow organ is, in particular, a vessel or a vessel system, for example, a blood vessel or a blood vessel system. The tool is, in particular, a vessel catheter, in particular what is known as a microcatheter.

According to at least one embodiment, a three-dimensional representation of the hollow organs is obtained or determined, in particular before generating the X-ray image. The angle, which the longitudinal direction of the tool encloses with the X-ray projection plane, is determined as a function of an orientation of the three-dimensional representation in respect of the X-ray projection plane and a location of the tool in the three-dimensional representation.

The three-dimensional representation may therefore be, in particular, a preoperative or pre-interventional 3D reconstruction or a corresponding 3D model. The three-dimensional representation may be generated, in particular, by a CT acquisition.

The hollow organ may be segmented in the three-dimensional representation. A user, in particular medical staff, may potentially estimate at which position of the three-dimensional representation or at which corresponding position of the hollow organ the tool is approximately located. The location of the tool in the three-dimensional representation is thereby approximately determined. Further, the location of the three-dimensional representation in respect of the X-ray projection plane is also known, so the angle of the tool or the longitudinal direction with the X-ray projection plane may be estimated. A more reliable localization may thus be achieved.

In particular, the three-dimensional representation of the hollow organ and the X-ray image are registered relative to one another, or the three-dimensional representation of the hollow organ and the X-ray image are obtained registered relative to one another.

According to at least one embodiment, the portion is a region of interest of a plurality of specified and, in particular, non-overlapping regions of interest of the X-ray image.

The plurality of specified regions of interest may be, for example, a plurality of grid cells or raster cells of the X-ray image. The X-ray image is therefore divided, in particular, into the plurality of regions of interest. The described method acts may be carried out for different regions of interest, in particular, until that region of interest is identified in which the tool may be localized as described.

The necessary computing effort may be reduced by the division of the X-ray image into the plurality of specified regions of interest.

According to at least one embodiment, the spatial frequency spectrum of the portion is determined using a local filter, wherein a position of the local filter defines a position of the portion in the X-ray image.

The width or extent of the local filter parallel to the X-ray projection plane therefore determines or defines the size of the portion. The isotropy or anisotropy of the local filter defines the form of the portion. The local filter may be a Gaussian filter or another local filter. In particular, the local filter may be convoluted with an operator for the Fourier transform in order to determine the spatial frequency spectrum of the portion.

The use of such a local filter makes it possible to dispense with prior division into regions of interest according to a corresponding raster or the like. However, it is also possible to combine both approaches with one another.

According to at least one embodiment, a modified X-ray image is displayed on a display device on the basis of the X-ray image, wherein the portion in the modified X-ray image is visually highlighted compared with the X-ray image if it is established when checking that the spatial frequency spectrum corresponds to the absorption characteristic of the tool.

The visual highlighting may take place, for example, by way of an altered brightness and/or color and/or a frame or another form of visual highlighting. The observer may in this way be quickly and intuitively pointed toward the position of interest in the X-ray image during the intervention.

According to at least one embodiment, the tool is embodied as a catheter for introducing into a blood vessel or as a catheter for introducing into another hollow organ. The catheter may be introduced directly into the hollow organ or blood vessel or, for example, through a further catheter. In the latter case, the tool is embodied, in particular, as what is known as a microcatheter. The further catheter may be introduced into the hollow organ or blood vessel and the microcatheter may be guided through the further catheter.

According to a further aspect, a data processing apparatus is disclosed. The data processing apparatus has at least one computing unit configured to carry out an method for localizing a tool in an X-ray image.

A computing unit may refer to a data processing device that includes a processor or processing circuit. The computing unit may therefore process data for carrying out computing operations. These potentially also include operations to carry out indicated instances of access to a data structure, for example, a look-up table (LUT).

The computing unit may include one or more computer(s), one or more microcontroller(s), and/or one or more integrated circuit(s), (e.g., one or more application-specific integrated circuit(s) (ASIC), one or more field-programmable gate array(s) (FPGA), and/or one or more system(s) on a chip (SoC)). The computing unit may also include one or more processor(s), for example one or more microprocessor(s), one or more central processing unit(s) (CPU), one or more graphics processing unit(s) (GPU) and/or one or more signal processor(s), in particular one or more digital signal processor(s) (DSP). The computing unit may also include a physical or a virtual network of computers or other of the units.

In certain embodiments, the computing unit includes one or more hardware and/or software interface(s) and/or one or more memory unit(s).

A memory unit may be embodied as a volatile data memory, for example, as a dynamic random access memory (DRAM) or static random access memory (SRAM), or as a non-volatile data memory, for example, as a read-only memory (ROM), as a programmable read-only memory (PROM), as an erasable programmable read-only memory (EPROM), as an electrically erasable programmable read-only memory (EEPROM), as a flash memory or flash EEPROM, as a ferroelectric random access memory (FRAM), as a magnetoresistive random access memory (MRAM), or as a phase-change random access memory (PCRAM).

According to a further aspect, an X-ray imaging system is disclosed that has a data processing apparatus.

The X-ray imaging system may be embodied, in particular, as an X-ray angiography system.

The X-ray imaging system has, in particular, an X-ray source and an X-ray detector as well as the at least one computing unit. The at least one computing unit may actuate the X-ray source to generate X-ray radiation and emit the radiation in the direction of a region for the arrangement of an object or patient to be examined. The X-ray detector is arranged to detect fractions of the X-ray radiation that have penetrated through the object and may generate corresponding detector signals and transfer them to the at least one computing unit. The at least one computing unit is configured to generate the X-ray image on the basis of the detector signals.

According to various embodiments, the X-ray imaging system also includes the tool as described herein.

Further embodiments of the X-ray imaging system follow directly from the various embodiments of the method, and vice versa. In particular, individual features and corresponding explanations as well as advantages in respect of the various embodiments relating to the method may be analogously transferred to corresponding embodiments of the X-ray imaging system. In particular, the X-ray imaging system is embodied or programmed for carrying out an method. In particular, the X-ray imaging system carries out the method.

According to a further aspect, a tool for a medical intervention on a hollow organ is disclosed. Along a specified longitudinal direction of the tool, the tool has a spatially changeable X-ray absorption strength according to a specified absorption characteristic.

The tool is embodied in various embodiments, in particular, as a catheter, for example, as a microcatheter.

Further embodiments of the tool may be found in the embodiments of the method for localizing a tool and the associated explanations.

According to a further aspect, a computer program with commands is disclosed. On execution of the commands by a data processing apparatus, the commands prompt the data processing apparatus to carry out a method for localizing a tool in an X-ray image.

The commands may be in the form, for example, of program code. The program code may be provided, for example, as binary code or Assembler and/or as a source code of a programming language, (e.g., C), and/or as a program script, (e.g., Python).

According to a further aspect, a computer-readable storage medium is disclosed that stores a computer program.

The computer program and the computer-readable storage medium may each be referred to as the computer program product with the commands.

Further features of the disclosure may be found in the claims, the figures, and the description of the figures. The features and combinations of features mentioned above in the description as well as the features and combinations of features mentioned below in the description of the figures and/or shown in the figures may be encompassed by the disclosure not only in the respectively disclosed combination but also in other combinations. In particular, embodiments and combinations of features, which do not have all features of an originally worded claim, may also be encompassed by the disclosure. Furthermore, embodiments and combinations of features that go beyond the combinations of features set out in the back references of the claims or which deviate from these may also be encompassed by the disclosure.

### BRIEF DESCRIPTION OF THE DRAWINGS

The disclosure is explained in more detail below on the basis of specific embodiments and associated schematic drawings. Identical or functionally identical elements may be provided with the same reference numerals in the figures. The description of identical or functionally identical elements may not necessarily be repeated in respect of different figures.

FIG. 1 depicts a schematic block representation of an embodiment of an X-ray imaging system.

FIG. 2 schematically shows an exemplary representation of a spatial change in the X-ray absorption strength of a tool.

FIG. 3 depicts an example of a schematic representation of a reference spatial frequency spectrum corresponding to the X-ray absorption strength in FIG. 2.

FIG. 4 schematically shows an exemplary representation of a spatial change in the X-ray absorption strength of a further tool.

FIG. 5 schematically shows an exemplary representation of a spatial change in the X-ray absorption strength of a further tool.

### DETAILED DESCRIPTION

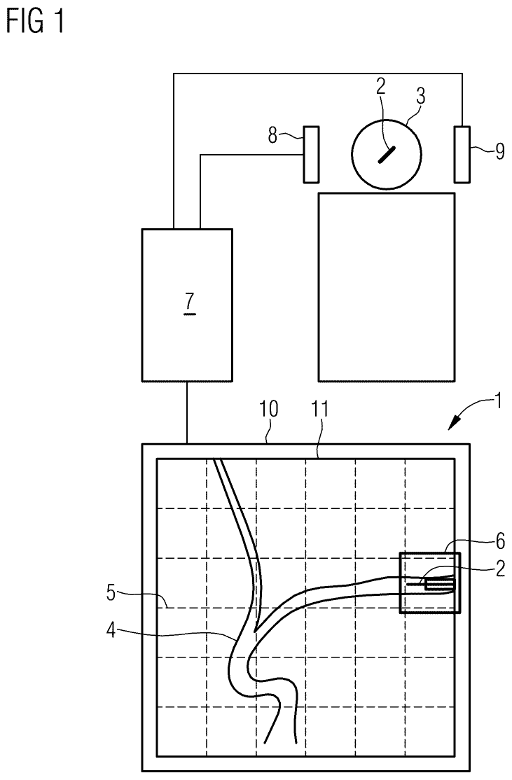

FIG. 1 schematically represents an embodiment of an X-ray imaging system 1 having a data processing apparatus 7 with at least one computing unit. The at least one computing unit is configured to carry out an embodiment of a method for localizing a tool 2 as described herein.

The X-ray imaging system 1 may include an X-ray source 8 and an X-ray detector 9 arranged on different opposing sides of an acquisition region for positioning of a patient 3. The X-ray imaging system 1 may have a display device 10, moreover, for displaying X-ray images and/or other items of information.

During an intervention, a tool 2, for example, may be introduced into the body of the patient 3. The tool 2 may be, for example, a microcatheter that is introduced into a blood vessel or another hollow organ 4 of the patient 3. In this state, the at least one computing unit 7 may actuate the X-ray source 8 to generate X-ray radiation and receive detector signals generated by the X-ray detector 9 accordingly in order to generate an X-ray image on the basis thereof that represents the tool 2 and, for example, the hollow organ 4 into which the tool 2 has been introduced. The at least one computing unit may identify a portion 6 of the X-ray image in which the tool 2 is located. For example, the at least one computing unit may display a modified X-ray image 11 on the display device 10 in that the portion 6 is visually highlighted compared to the original X-ray image.

In order to determine the portion 6 as the position of the tool 2, the at least one computing unit may determine, in particular, a spatial frequency spectrum of the portion 6. The at least one computing unit may then check whether the spatial frequency spectrum corresponds to the absorption characteristic of the tool, with the absorption characteristic of the tool defining a spatially changeable X-ray absorption strength 12 of the tool along a specified longitudinal direction. If it is established that the spatial frequency spectrum of the portion 6 corresponds to the absorption characteristic of the tool 2, the portion 6 may thus be determined as the position of the tool 2.

For example, a raster 5 may be specified that divides the X-ray image into a plurality of regions of interest and the portion 6 may be one of these regions of interest. The at least one computing unit may check the individual regions of interest of the raster 5 as described in order to identify the portion 6 in which the tool is arranged. In addition or instead of the raster 5, a local filter, (e.g., a Gaussian filter), may also be used to spatially limit the involved Fourier transform for determining the spatial frequency spectrum of the portion 6.

The X-ray imaging system 1 is embodied, in particular, as a 2D X-ray imaging system, (e.g., as C-arm-based X-ray imaging system 1). In other words, the X-ray source 8 and the X-ray detector 9 may be attached to a C-arm of the X-ray imaging system 1, so the X-ray projection plane may be purposefully set or changed. This is not imperative for the implementation of the method, however.

A modulation transfer function, for example, may be provided that describes how spatial domain frequencies of the material absorption in the object domain are transferred into spatial frequencies of the image brightness in the image domain.

The tool 2, in particular the microcatheter, is provided in such a way that a changeable X-ray absorption strength 12 is present in the longitudinal direction, for example, a periodic or recurring change in the X-ray absorption strength 12. This may be achieved, for example, in that the material composition is changed along the longitudinal direction. For example, a different metal concentration in corresponding composite materials may be used or the surface of the tool 2 may be coated in different regions with highly absorbent metals or the like and/or a fraction of plastics materials or more or fewer X-ray-transparent fibers, etc. in a corresponding composite may be modulated.

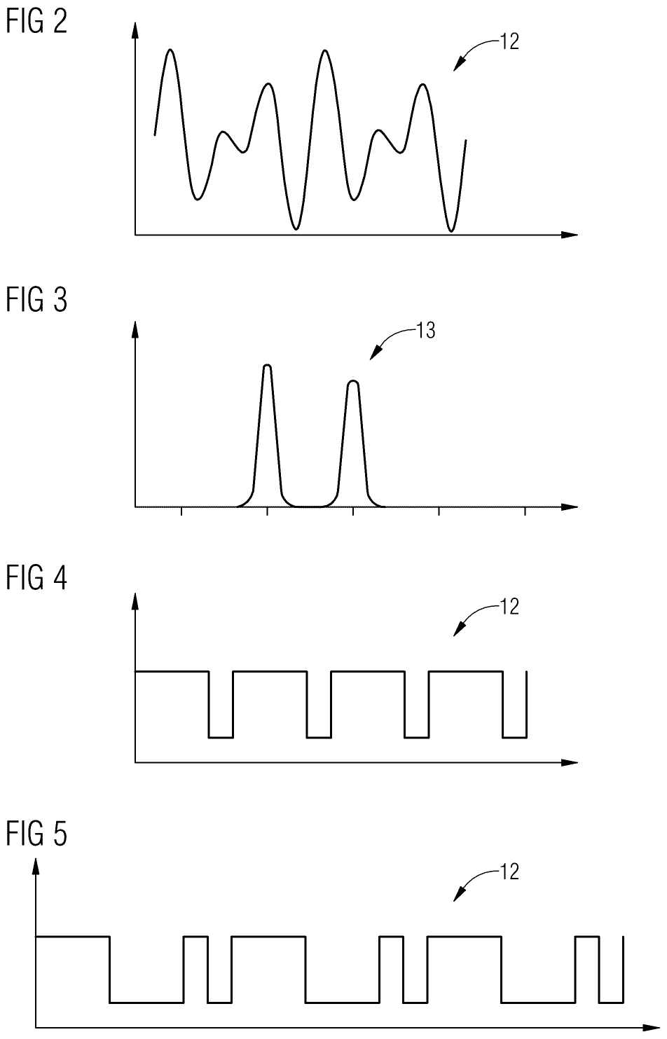

FIG. 2 schematically represents a non-limiting example of a spatially changeable X-ray absorption strength 12. This is, in particular, an overlay of two harmonic oscillations with frequencies of 2 or 3 in arbitrary units over a length of 4π. The oscillation with the greater frequency of 3 therefore occurs six times in the length shown; the oscillation with the lower frequency of 2 occurs four times. FIG. 3 schematically represents a corresponding reference spatial frequency spectrum 13 with peaks at the points 2 and 3 respectively.

FIG. 4 and FIG. 5 schematically represent other exemplary periodic characteristic curves of the X-ray absorption strength 12, which are in the form of discrete stages. The example of FIG. 2 represents the characteristic curve of the X-ray absorption strength 12 by way of a simple rectangular signal with a pulse duty factor of more than 50%, for example of approximately 75%. The example of FIG. 5 represents a slightly more complex sequence of rectangular pulses with two different pulse durations and period durations.

Depending on the size of the available space along the longitudinal direction, in order to implement the changeable X-ray absorption strength, more or less complex characteristic curves, (e.g., periodic characteristic curves), of the X-ray absorption strength 12 may thus be achieved to enable an optimally clear and nevertheless simple localization of the tool 2.

It may be assumed, for example, that the longitudinal direction of the tool 2 is substantially perpendicular to the X-ray projection direction, i.e., lies in the X-ray projection plane or parallel to it. However, deviations of up to 30° or also up to 45° for the angle, which the longitudinal direction encloses with the X-ray projection plane, may also be accepted. The corresponding frequency characteristic curves or positions of peaks in the reference spatial frequency spectrum are subject to the correspondingly likewise tolerances or deviations.

The spatial frequency spectrum in the portion 6 has, if the tool 2 is located there, a characteristic that is easy to identify, (e.g., with one or more peaks or two or more peaks with defined spacings). This enables very sensitive localization of the tool 2 in the X-ray image. The MTF of the image chain may likewise be taken into account, in particular in order to compare the relative heights of the various peaks and/or their position relative to each other and/or their form in the frequency domain.

Depending on how accurately the angle, which the longitudinal direction encloses with the X-ray projection plane, is known or may be localized, the more accurately the peaks may be localized in the frequency spectrum.

The orientation of the tool 2 relative to the X-ray projection plane may be estimated, for example using preoperative CT volumes. It is also possible to generate a first and a second X-ray image and to carry out the described analysis in both X-ray images. The first and the second X-ray images then correspond to different X-ray projection directions, e.g., X-ray projection directions that are perpendicular to one another. The orientation of the longitudinal direction in respect of the X-ray projection plane may be estimated by way of the shift in the peak positions or the change in the spacings of the individual peaks and/or the change in the form of the peaks in the respective spatial frequency spectra according to the first and the second X-ray images. For example, on this basis the X-ray projection plane may be set in such a way that the longitudinal direction lies in the X-ray projection plane and a third X-ray image may accordingly be acquired in order to repeat the exact localization.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for localizing a tool in an X-ray image, wherein the tool has an X-ray absorption strength that is spatially changeable along a specified longitudinal direction of the tool according to a specified absorption characteristic, the method comprising:
determining a spatial frequency spectrum of a portion of the X-ray image;
checking whether the spatial frequency spectrum corresponds to the specified absorption characteristic of the tool; and
determining the portion of the X-ray image as a position of the tool when the spatial frequency spectrum corresponds to the specified absorption characteristic of the tool.

2. The method of claim 1, wherein, according to the specific absorption characteristic, a spatial change in the X-ray absorption strength along the specified longitudinal direction recurs or recurs up to a differing amplitude.

3. The method of claim 1, wherein, according to the specific absorption characteristic, a spatial change in the X-ray absorption strength along the specified longitudinal direction has a characteristic frequency at which a reference spatial frequency spectrum of the spatial change in the X-ray absorption strength along the specified longitudinal direction has a peak value, and
wherein the checking comprises verifying whether the spatial frequency spectrum has a peak value at a point that corresponds to the characteristic frequency within a specified tolerance range.

4. The method of claim 1, wherein, according to the specific absorption characteristic, a spatial change in the X-ray absorption strength along the specified longitudinal direction has two characteristic frequencies at which a reference spatial frequency spectrum of the spatial change in the X-ray absorption strength along the specified longitudinal direction has one peak value respectively, and
wherein the checking comprises verifying whether the spatial frequency spectrum has one peak value respectively at two points whose spacing from one another corresponds to a spacing of the two characteristic frequencies from one another within a specified tolerance range.

5. The method of claim 4, wherein the checking comprises verifying whether a relationship of the peak values at the two points of the spatial frequency spectrum corresponds to a relationship of the peak values in a case of the two characteristic frequencies of the reference spatial frequency spectrum.

6. The method of claim 1, wherein the checking is carried out as a function of a specified modulation transfer function.

7. The method of claim 1, further comprising:
generating a further X-ray image that represents the tool in a calibration environment; and
determining the specified absorption characteristic based on the further X-ray image.

8. The method of claim 7, further comprising:
obtaining or determining an angle that the specified longitudinal direction of the tool encloses with a specified X-ray projection plane when generating the X-ray image,
wherein the checking is carried out as a function of the angle.

9. The method of claim 8, wherein the X-ray image maps a hollow organ,
wherein the tool is arranged in the hollow organ,
wherein a three-dimensional representation of the hollow organ is obtained or determined, and
wherein the angle is determined as a function of an orientation of the three-dimensional representation in respect of the specified X-ray projection plane and a location of the tool in the three-dimensional representation.

10. The method of claim 8, further comprising:
displaying a modified X-ray image on a display device based on the X-ray image,
wherein the portion is visually highlighted when the spatial frequency spectrum corresponds to the specific absorption characteristic of the tool.

11. The method of claim 1, further comprising:
obtaining or determining an angle that the specified longitudinal direction of the tool encloses with a specified X-ray projection plane when generating the X-ray image,
wherein the checking is carried out as a function of the angle.

12. The method of claim 11, wherein the X-ray image maps a hollow organ,
wherein the tool is arranged in the hollow organ,
wherein a three-dimensional representation of the hollow organ is obtained or determined, and
wherein the angle is determined as a function of an orientation of the three-dimensional representation in respect of the specified X-ray projection plane and a location of the tool in the three-dimensional representation.

13. The method of claim 1, wherein the portion is a region of interest of a plurality of specified regions of interest of the X-ray image, or
wherein the spatial frequency spectrum of the portion is determined using a local filter, wherein a position of the local filter defines the position of the portion in the X-ray image.

14. The method of claim 1, further comprising:
displaying a modified X-ray image on a display device based on the X-ray image,
wherein the portion is visually highlighted when the spatial frequency spectrum corresponds to the specific absorption characteristic of the tool.

15. The method of claim 1, wherein the tool is a catheter for introducing into a blood vessel or another hollow organ.

16. A data processing apparatus comprising:
at least one computing unit configured to:
determine a spatial frequency spectrum of a portion of an X-ray image;
check whether the spatial frequency spectrum corresponds to a specified absorption characteristic of a tool in the X-ray image, wherein the tool has an X-ray absorption strength that is spatially changeable along a specified longitudinal direction of the tool according to the specified absorption characteristic; and
determine the portion of the X-ray image as a position of the tool when the spatial frequency spectrum corresponds to the specified absorption characteristic of the tool.

17. An X-ray imaging system comprising:
a data processing apparatus having at least one computing unit configured to:
determine a spatial frequency spectrum of a portion of an X-ray image;
check whether the spatial frequency spectrum corresponds to a specified absorption characteristic of a tool in the X-ray image, wherein the tool has an X-ray absorption strength that is spatially changeable along a specified longitudinal direction of the tool according to the specified absorption characteristic; and
determine the portion of the X-ray image as a position of the tool when the spatial frequency spectrum corresponds to the specified absorption characteristic of the tool.
